(19) United States
(12) Patent Application Publication
(10) Pub. No.: US 2026/0108875 A1
(43) Pub. Date: Apr. 23, 2026

(54) ELECTRONIC TEST RESULT DETERMINATION AND CONFIRMATION

(71) Applicants: RTX BBN Technologies, Inc., Cambridge, MA (US); Purdue Research Foundation, West Lafayette, IN (US)

(72) Inventors: Aaron Adler, Towson, MD (US); Bryan Bartley, Friday Harbor, WA (US); Mike Gavin, Princeton, NJ (US); Jordan Seville, Delran, NJ (US); Darby McChesney, Lawrenceville, NJ (US); Frank M. LaDuca, Warrensburg, NY (US); Jiangshan Wang, West Lafayette, IN (US); Andres Dextre, West Lafayette, IN (US); Mohit Verma, West Lafayette, IN (US)

(21) Appl. No.: 19/427,431

(22) Filed: Dec. 19, 2025

Related U.S. Application Data
(63) Continuation of application No. 17/576,977, filed on Jan. 16, 2022.
(60) Provisional application No. 63/138,310, filed on Jan. 15, 2021.
(60) Provisional application No. 63/138,312, filed on Jan. 15, 2021.
(60) Provisional application No. 63/138,314, filed on Jan. 15, 2021.
(60) Provisional application No. 63/138,316, filed on Jan. 15, 2021.
(60) Provisional application No. 63/138,318, filed on Jan. 15, 2021.
(60) Provisional application No. 63/138,320, filed on Jan. 15, 2021.
(60) Provisional application No. 63/138,321, filed on Jan. 15, 2021.
(60) Provisional application No. 63/138,323, filed on Jan. 15, 2021.
(60) Provisional application No. 63/138,337, filed on Jan. 15, 2021.
(60) Provisional application No. 63/138,341, filed on Jan. 15, 2021.
(60) Provisional application No. 63/148,527, filed on Feb. 11, 2021.

(51) Int. Cl. B01L 3/00; B01L 7/00; C12Q 1/6806; C12Q 1/6844; C12Q 1/6848; C12Q 1/6888; C12Q 1/70; G01J 3/46; G01J 3/52; G01K 7/02; G01K 7/22; G01N 1/44; G01N 21/78; G01N 33/52

(52) U.S. Cl. CPC B01L 3/5023; B01L 7/00; C12Q 1/6806; C12Q 1/6844; C12Q 1/6848; C12Q 1/6888; C12Q 1/70; C12Q 1/701; G01J 3/524; G01K 7/02; G01K 7/22; G01N 1/44; G01N 21/78; G01N 33/526; B01L 2200/04; B01L 2200/0689; B01L 2200/10; B01L 2200/16; B01L 2300/0681; B01L 2300/069; B01L 2300/0809; B01L 2300/0825; B01L 2300/0832; B32B 2250/04; C12Q 2600/112; G01J 2003/467

(57) ABSTRACT

A technology is described for a system for identifying a colorimetric test result from a target nucleotide test performed on a solid phase substrate. The system can comprise a sensor configured to detect a spectrum of color wavelengths. The system can comprise one or more processors. The one or more processors can be configured to: receive color wavelength data; determine a wavelength threshold for providing a target nucleotide positive test result; identify whether the color wavelength data meets or exceeds the wavelength threshold for providing a target nucleotide positive test result; and generate a result indicator indicating either a target nucleotide positive or target nucleotide negative test result.

### CROSS-REFERENCE TO RELATED APPLICATIONS

This application is a continuation of U.S. Application No. Ser. No. 17/576,977 filed Jan. 16, 2022, which claims the benefit of U.S. Provisional Patent Application Ser. Nos. 63/138,310 filed Jan. 15, 2021, 63/138,312 filed Jan. 15, 2021, 63/138,314 filed Jan. 15, 2021, 63/138,316 filed Jan. 15, 2021, 63/138,318 filed Jan. 15, 2021, 63/138,320 filed Jan. 15, 2021, 63/138,321 filed Jan. 15, 2021, 63/138,323 filed Jan. 15, 2021, 63/138,337 filed Jan. 15, 2021, 63/138,341 filed Jan. 15, 2021, 63/148,527 filed Feb. 11, 2021, the entire contents of each of which are incorporated herein by reference.

### BACKGROUND

A rapid diagnostic test (RDT) for infectious diseases typically refers to lateral-flow immunochromatographic tests used to detect infections. In some cases, but not always, an RDT may constitute a point-of-care (POC) test. For example, some molecular diagnostics, e.g., polymerase chain reaction (PCR), may only be available at a central laboratory that has access to top bench equipment for performance at a prohibitive cost. Consequently, many RDTs that may be used at a hospital and subsequently shipped for testing are impracticable in other use cases including large-scale (e.g., a concert, mall, university), medium scale (day care, restaurant, café), retail health, and at the home.

Even when a RDT is available in a POC setting, the use and interpretation of the test can be complicated and confusing to many users. The sensitivity of rapid antigen tests for influenza is low and should not be used to dictate therapeutical decisions. Rapid tests for rotavirus and norovirus are less sensitive than molecular tests. Therefore, rapid diagnostic tests that are applicable at the point of care are in high demand.

### BRIEF DESCRIPTION OF THE DRAWINGS

Features and advantages of the disclosure will be apparent from the detailed description which follows, taken in conjunction with the accompanying drawings, which together illustrate, by way of example, features of the disclosure; and, wherein:

FIG. 1 depicts a flowchart for identifying a colorimetric test result from a pathogen test performed on a solid phase substrate in accordance with an example embodiment;

FIG. 2 depicts a pathogen test performed on a solid phase substrate in accordance with an example embodiment;

FIG. 3 depicts a flowchart for generating a confidence level in accordance with an example embodiment;

FIG. 4 depicts a system for identifying a colorimetric test result from a pathogen test performed on a solid phase substrate in accordance with an example embodiment;

FIG. 5 depicts a method of identifying a test result of a loop-mediated isothermal amplification (LAMP) reaction on a solid phase reaction medium in accordance with an example embodiment;

FIG. 6 depicts a flowchart of a machine readable storage medium having instructions embodied thereon for identifying a test result of a loop-mediated isothermal amplification (LAMP) reaction in accordance with an example embodiment; and

FIG. 7 illustrates a computing system that includes a data storage device in accordance with an example embodiment.

Reference will now be made to the exemplary embodiments illustrated, and specific language will be used herein to describe the same. It will nevertheless be understood that no limitation of the scope of the invention is thereby intended.

### DETAILED DESCRIPTION

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular examples only and is not intended to be limiting. The same reference numerals in different drawings represent the same element. Numbers provided in flow charts and processes are provided for clarity in illustrating steps and operations and do not necessarily indicate a particular order or sequence.

Furthermore, the described features, structures, or characteristics can be combined in any suitable manner in one or more embodiments. In the following description, numerous specific details are provided, such as examples of layouts, distances, network examples, etc., to provide a thorough understanding of various invention embodiments. One skilled in the relevant art will recognize, however, that such detailed embodiments do not limit the overall technological concepts articulated herein, but are merely representative thereof.

As used in this written description, the singular forms “a,” “an” and “the” include express support for plural referents unless the context clearly dictates otherwise. Thus, for example, reference to “a sensor” includes a plurality of such sensors.

Reference throughout this specification to “an example” means that a particular feature, structure, or characteristic described in connection with the example is included in at least one embodiment of the present invention. Thus, appearances of the phrases “in an example” or “an embodiment” in various places throughout this specification are not necessarily all referring to the same embodiment.

As used herein, a plurality of items, structural elements, compositional elements, and/or materials can be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present technology can be referred to herein along with alternatives for the various components thereof. It is understood that such embodiments, examples, and alternatives are not to be construed as de facto equivalents of one another, but are to be considered as separate and autonomous representations under the present disclosure.

Furthermore, the described features, structures, or characteristics can be combined in any suitable manner in one or more embodiments. In the following description, numerous specific details are provided, such as examples of layouts, distances, network examples, etc., to provide a thorough understanding of invention embodiments. One skilled in the relevant art will recognize, however, that the technology can be practiced without one or more of the specific details, or with other methods, components, layouts, etc. In other instances, well-known structures, materials, or operations may not be shown or described in detail to avoid obscuring aspects of the disclosure.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term in this written description, like “comprising” or “including,” it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The terms “coupled” and “connected” can be used interchangeably and refer to a relationship between items or structures that are either directly or indirectly connected in an electrical or nonelectrical manner. “Directly coupled” or “directly connected” objects or elements are in physical contact with one another. In this written description, recitation of “coupled” or “connected” provides express support for “directly coupled” or “directly connected” and vice versa. Objects described herein as being “adjacent to” each other may be in physical contact with each other, in close proximity to each other, or in the same general region or area as each other, as appropriate for the context in which the phrase is used.

Occurrences of the phrase “in one embodiment,” or “in one aspect,” herein do not necessarily all refer to the same embodiment or aspect.

As used herein, comparative terms such as “increased,” “decreased,” “better,” “worse,” “higher,” “lower,” “enhanced,” “maximized,” “minimized,” and the like refer to a property of a device, component, or activity that is measurably different from other devices, components, or activities in a surrounding or adjacent area, in a single device or in multiple comparable devices, in a group or class, in multiple groups or classes, or as compared to the known state of the art. For example, a sensor with “increased” sensitivity can refer to a sensor in a sensor array which has a lower level or threshold of detection than one or more other sensors in the array. A number of factors can cause such increased sensitivity, including materials, configurations, architecture, connections, etc.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. However, it is to be understood that even when the term “about” is used in the present specification in connection with a specific numerical value, that support for the exact numerical value recited apart from the “about” terminology is also provided.

Numerical amounts and data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 1.5, 2, 2.3, 3, 3.8, 4, 4.6, 5, and 5.1 individually.

This same principle applies to ranges reciting only one numerical value as a minimum or a maximum. Furthermore, such an interpretation should apply regardless of the breadth of the range or the characteristics being described.

### Example Embodiments

An initial overview of technology embodiments is provided below and then specific technology embodiments are described in further detail later. This initial summary is intended to aid readers in understanding the technology more quickly but is not intended to identify key features or essential features of the technology nor is it intended to limit the scope of the claimed subject matter.

Viral pathogens can spread from pre-symptomatic and asymptomatic individuals. Individuals can remain infectious for up to ten days in moderate cases, and up to two weeks in severe cases. One diagnosis method is by real-time reverse transcription polymerase chain reaction (rRT-PCR) from a nasopharyngeal swab, but results are not usually available for at least a few hours to about a few days. Delays in testing can often led to delays in treatment and delays in mitigating the risk of further spreading the disease.

The utility of a rapid diagnostic test for pathogens is mitigated by the access to the expensive laboratories used to process test results. In many cases, a person afflicted with a disease would not know test results until after: (1) the person has submitted a biological sample at a healthcare facility; (2) the healthcare facility has processed and shipped the biological sample to a laboratory; (3) the laboratory has tested the sample (which can be limited by supplies and personnel); (4) the laboratory has sent the results back to the healthcare facility; (5) the medical personnel at the healthcare facility have analyzed the results; and (6) the medical personnel communicate the test results to the person afflicted with the disease. A rapid diagnostic test that is also a point of care (POC) test (i.e. a short test time with decision-affecting results that can be delivered in the same encounter between a diseased person and healthcare personnel) would provide a further benefit.

When a timely test result can be obtained from a rapid diagnostic test, a POC test can provide an additional enhancement when errors in accuracy or undue uncertainty from the test result are minimized. Some potential sources of testing error include when a test result falsely indicates that a diseased person does not have a disease (a false negative) or falsely indicates that a healthy person does have a disease (a false positive). Additional sources of testing error can include: (i) a clinician error in interpreting the test results, or (ii) a user error in interpreting the test results. These clinician and user errors can be caused by inadequate knowledge, poor critical thinking skills, a lack of competency, issues in data gathering, failing to synthesize information, and the like. In some cases, testing equipment errors can lead to clinician/user errors, and clinician/user errors can also lead to testing equipment errors. As a result, a rapid diagnostic test that can be used in a POC setting while also minimizing testing equipment errors and clinician/user interpretation errors would also be beneficial.

Even when a timely test result can be obtained and the foregoing errors are minimized, the test can provide an uncertain result when the test is inconclusive and an additional test or a different test is used to provide additional information. Therefore, a rapid diagnostic test in a POC setting that minimized testing equipment errors, minimized clinician/user interpretation errors, and minimized uncertain results would be desirable.

In one disclosure embodiment, systems for identifying a colorimetric test result from a pathogen test performed on a solid phase substrate can comprise a sensor configured to detect a spectrum of color wavelengths, and one or more processors. The one or more processors can be configured to receive color wavelength data. The one or more processors can be configured to determine a wavelength threshold for providing a pathogen positive test result. As used herein, a wavelength threshold can be a threshold based on intensity levels at one or more wavelengths. The one or more processors can be configured to identify whether the color wavelength data meets or exceeds the wavelength threshold for providing a pathogen positive test result. The one or more processors can be configured to generate a result indicator indicating either a pathogen positive or pathogen negative test result.

In another disclosure embodiment, methods of identifying a test result of a loop-mediated isothermal amplification (LAMP) reaction on a solid phase reaction medium can comprise detecting, using a sensor component, a spectrum of color wavelengths. The method can further comprise receiving, at one or more processors, color wavelength data from the sensor component. The method can further comprise determining, at the one or more processors, whether the color wavelength data meets or exceeds the wavelength threshold for providing a positive test result. The method can further comprise generating, at the one or more processors, a result indicator indicating either a positive or negative test result. The method can further comprise displaying, at a user interface, a test result based on the result indicator.

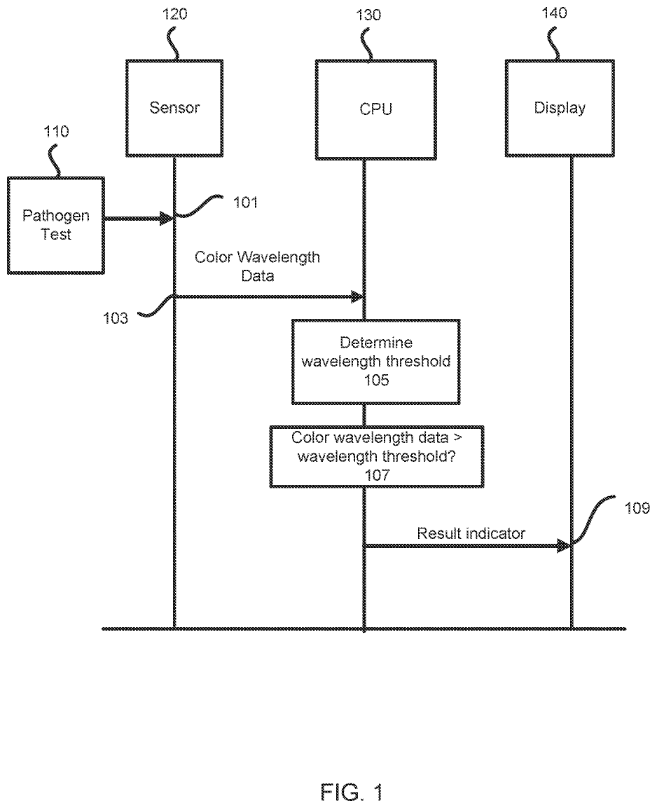

In one embodiment, as illustrated with reference to FIG. 1, a system for identifying a colorimetric test result from a pathogen test performed on a solid phase substrate can comprise a sensor 120 configured to detect a spectrum of color wavelengths from a pathogen test 110, as shown in operation 101.

In one aspect, the system can further comprise one or more processors 130 or a CPU 130. In one example, the one or more processors 130 can be configured to receive color wavelength data, as shown in operation 103. In one example, the color wavelength data can include data received by a sensor 120 including one or more of a photoconductive sensor, a photovoltaic sensor, a photodiode sensor, a phototransistor sensor, or combinations thereof a photoresistor, a photodiode array, a charge-coupled device (CCD) camera, a complementary metal-oxide semiconductor (CMOS) camera, the like, or combinations thereof.

In another example, the one or more processors 130 can be configured to determine a wavelength threshold for providing a pathogen positive test result, as shown in operation 105. In another example, the one or more processors 130 can be configured to identify whether the color wavelength data meets or exceeds the wavelength threshold for providing a pathogen positive test result, as shown in operation 107. In yet another example, the one or more processors 130 can be configured to generate a result indicator indicating either a pathogen positive or pathogen negative test result, as shown in operation 109. The one or more processors 130 can be configured to send the result indicator to a display 140, as shown in operation 109.

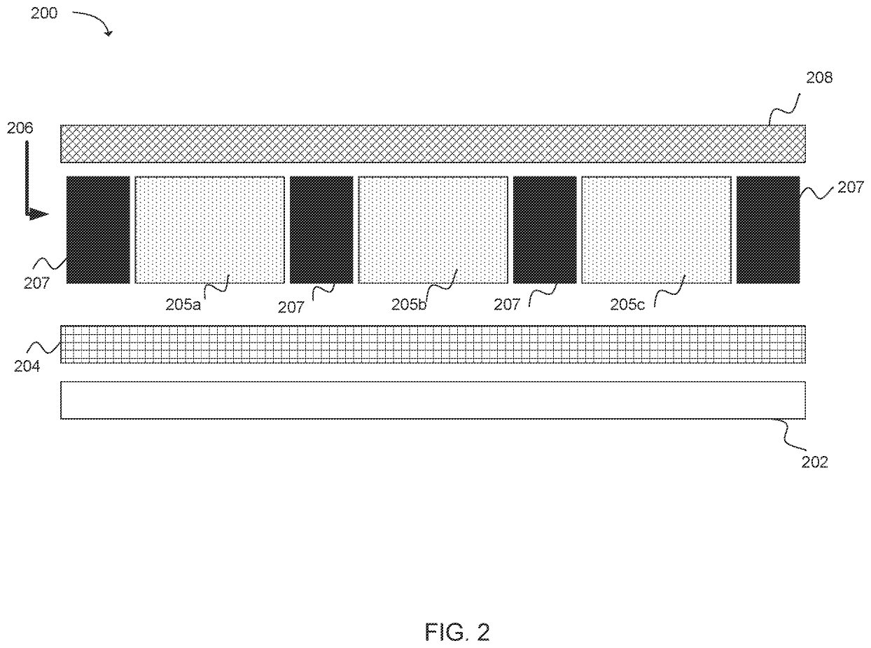

In another aspect, as illustrated in FIG. 2, a pathogen test system 200 can comprise one or more of a substrate 202, an adhesive layer 204, a reaction layer 206 comprising one or more reaction sections 205 a, 205 b and 205 c, a plurality of spacing layers 207 separating the one or more reaction sections 205 a-c, or a spreading layer 208. In one aspect, a sensor can be configured to detect color wavelength data from the one or more reaction sections 205 a, 205 b and 205 c from the reaction layer 206.

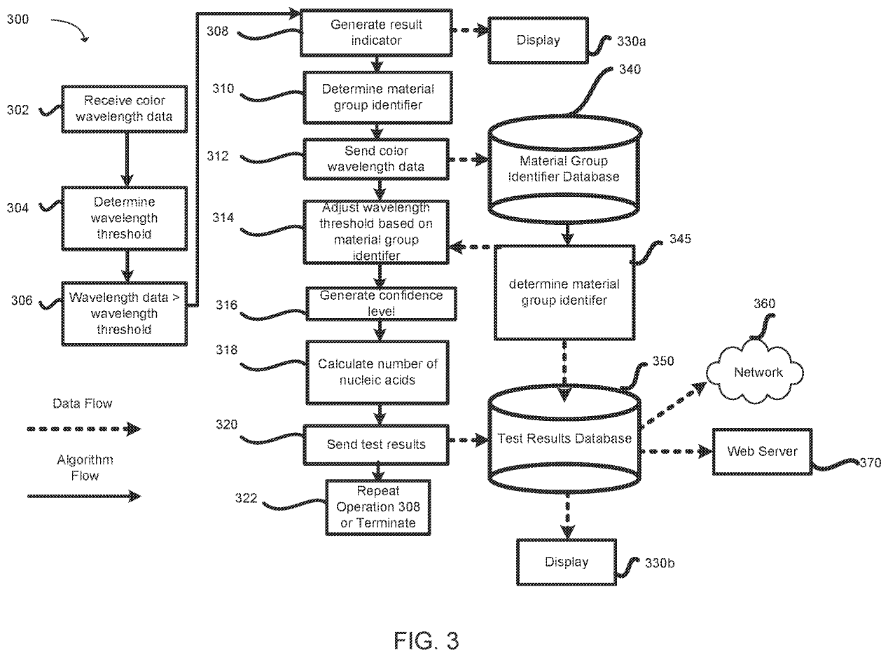

In another aspect, as depicted in the flowchart 300 in FIG. 3, the one or more processors can be configured to receive color wavelength data from one or more sensors, as shown in operation 302. The color wavelength data can be received from discrete sections of a pathogen test (e.g., reaction sections 205 a, 205 b or 205 c), wherein each discrete section can be directed to the same pathogen or different pathogens. The one or more processors can be configured to determine a wavelength threshold, as shown in operation 304. The one or more processors can be configured to determine that the color wavelength data meets or exceeds the color wavelength threshold, as shown in operation 306. The one or more processors can be configured to generate the test result indicator, as shown in operation 308. The one or more processors can be configured to send the test result indicator to a display 330 a.

A material group identifier can be used to identify the characteristics of reaction layers associated with a manufacturing group. In another aspect, the one or more processors can be configured to determine a material group identifier, as shown in operation 310. The material group identifier in operation 310 can be based on one or more of an average color wavelength of a material group, a median color wavelength of the material group, a variance of the color wavelength of the material group, a manufacturing date and time of the material group, one or more reagent types of the material group, or one or more solid-phase reaction medium types of the material group, the like, or combinations thereof.

The one or more processors can be configured to send the color wavelength data to a material group identifier database 340, as shown in operation 312. The material group identifier database 340 can be configured to determine the material group identifier based on crowd-sourced information, as shown in operation 345. The material group identifier database 340 can send the material group identifier to the one or more processors. The material group identifier database 340 can send the material group identifier to a test results database 350.

The material group identifier database can exchange information from multiple devices. The material group identifier can use the collected information to constantly update the determination of the material group identifier by the material group identifier database when color wavelength data is received from a device (e.g., when operation 345 is performed).

In another aspect, the one or more processors can be configured to adjust the wavelength threshold based on the material group identifier, as shown in operation 314. The one or more processors can be configured to generate a confidence level, as shown in operation 316. The one or more processors can be configured to calculate a number of nucleic acids, as shown in operation 318. The one or more processors can be configured to send the test results to the test results database 350 as shown in operation 320, or repeat operation 308 or terminate, as shown in operation 322.

In one aspect, operation 308 can be repeated when the confidence level is below a selected threshold. For example, when the confidence level is less than about 95%, then operation 308 can be repeated through an iterative process until the confidence level has reached the threshold of 95%. In another aspect, the operation 308 can be repeated until the operation has been repeated a selected number of times. For example, when operation 308 has been repeated about 5 times, then the process can terminate.

In another aspect, the test results can be accessed from the test results database 350 via a display 330 b, a network 360, or a web server 370.

In some cases, the one or more processors can adjust the wavelength threshold based on the material group identifier without sending or receiving information to the material group identifier database 340. In this case, the material group can be included with the device.

In another aspect, the one or more processors can be configured to adjust the wavelength threshold based on a material group identifier. In one aspect, the material group identifier can be based on one or more of: an average color wavelength of a material group, a median color wavelength of the material group, a variance of the color wavelength of the material group, a manufacturing date and time of the material group, one or more reagent types of the material group, or one or more solid-phase reaction medium types of the material group, the like, or combinations thereof.

In another aspect, the one or more processors can be configured to generate the material group identifier from color wavelength data aggregated from crowd-sourced data. In one aspect, the crowd-sourced data can be associated with a plurality of users associated with the pathogen test. In one aspect, a confidence level of can be calculated from metadata associated with color wavelength data including one or more of a ratio of the number of aggregated test results from a specific material group identifier to the number of total test results from the specific group identifier; a variance of the aggregated test results associated with the specific material group identifier; a date and time when the aggregated test results are received from a user for each material group identifier, the like, or combinations thereof.

In another aspect, the one or more processors can be configured to adjust the wavelength threshold using color wavelength data having a wavelength from about 500 nm to about 565 nm. In one example, the color wavelength data can be adjusted based on a colorimetric range associated with a pH-sensitive dye (e.g., phenol red).

In another aspect, the one or more processors can be configured to calculate a number of nucleic acid copies based on one or more of the color wavelength data, the material group identifier, a color change time, or a rate of color change time. In one example, the number of nucleic acid copies can be calculated using data associated with the material group identifier including, but not limited to, an average color wavelength of a material group, a median color wavelength of the material group, a variance of the color wavelength of the material group, a manufacturing date and time of the material group, one or more reagent types of the material group, or one or more solid-phase reaction medium types of the material group.

In another example, the color change time can be the time between the initialization of a LAMP reaction and a positive test result. In one example, when the color change time for a material group averages about 30 minutes, and the color change time for the pathogen test is about 20 minutes, then the concentration of the pathogen can be higher than the average concentration for the average of the material group. In another example, when the change time for the pathogen test is about 40 minutes, then the concentration of the pathogen can be lower than the average concentration for the average of the material group. The concentration of the pathogen can be calibrated against the material group to provide an approximate number of copies of nucleic acid per volume.

In another aspect, the one or more processors can be configured to generate a confidence level using the color wavelength data and the material group identifier. In one example, a confidence level can be calculated using data associated with the material group identifier including, but not limited to, an average color wavelength of a material group, a median color wavelength of the material group, a variance of the color wavelength of the material group, a manufacturing date and time of the material group, one or more reagent types of the material group, or one or more solid-phase reaction medium types of the material group.

In yet another aspect, the one or more processors can be further configured to receive the color wavelength data for discrete sections (e.g., reaction sections 205 a, 205 b or 205 c) of the pathogen test. In one example, the color wavelength data can be received from a first reaction section of a pathogen test and a second reaction section of a pathogen test, wherein the first reaction section and the second reaction section can be configured to test for the same pathogens or different pathogens.

In yet another aspect, the one or more processors can be further configured to determine the wavelength threshold for providing a pathogen positive test result based on the color wavelength data for discrete sections of the pathogen test. In one example, a first reaction section of the pathogen test can be configured to have a first range of color wavelength data and a second reaction section of the pathogen test can be configured to have a second range of color wavelength data. In another example, a first reaction section of the pathogen test can be configured to the same range of color wavelength data as the second reaction section of the pathogen test.

In yet another aspect, the one or more processors can be further configured to identify whether the color wavelength data meets or exceeds the threshold for providing the pathogen positive test result. In one example, the threshold for the color wavelength data can be the same for each discrete section. When the threshold is the same for the discrete sections, the additional data can provide a positive or negative test result with additional certainty compared to a test result based on color wavelength data from one section.

In another example, the threshold for the color wavelength data can be different for each discrete section (e.g., reaction sections 205 a, 205 b or 205 c). In this example, the threshold for each discrete section can be based on a confidence level. In one example, a first threshold, when met or exceeded, may provide a confidence level of about 95%, while a second threshold, when met or exceeded, may provide a confidence level of about 99%. The confidence level for each section can be based on the quality of color wavelength data obtained. In one example, the quality of color wavelength obtained can differ based on the type of colorimetric indicator used in the pathogen test.

In yet another aspect, the one or more processors can be further configured to generate a confidence level for the pathogen positive test result based on the color wavelength data received from the discrete sections of the pathogen test (e.g., reaction sections 205 a, 205 b or 205 c).

In yet another aspect, the one or more processors can be further configured to: receive the color wavelength data for discrete sections of the pathogen test (e.g., reaction sections 205 a, 205 b or 205 c); determine an additional wavelength threshold for providing a different pathogen positive test result based on the color wavelength data for discrete sections of the pathogen test, and generate an additional result indicator indicating either an additional pathogen positive or additional pathogen negative test result when the discrete sections of the pathogen test are targeted to different pathogens. In one example, a first section of the pathogen test can be targeted to SARS-CoV-2 and the second section of the pathogen test can be targeted to influenza. In one aspect, the confidence level for a positive or negative test result for a first pathogen can be based on the confidence level for a positive or negative test result for a second pathogen. For example, a confidence level of about 90% for a positive test for influenza can negatively affect the confidence level for a positive test result for SARS-CoV-2.

In yet another aspect, the sensor can comprise an RGB sensor configured to generate RGB values or a CMYK sensor configured to generate CMYK values. In one example, the one or more processor can be configured to receive the RGB values from the RGB sensor. The one or more processors can be configured to determine a wavelength threshold for providing a pathogen positive test result based on the RGB values. The one or more processors can be configured to identify whether the color wavelength data meets or exceeds the wavelength threshold for providing a pathogen positive test result based on the RGB values. The one or more processors can be configured to generate a result indicator indicating either a pathogen positive or pathogen negative test result based on the RGB values.

In one example, the one or more processor can be configured to receive the CMYK values from the CMYK sensor. The one or more processors can be configured to determine a wavelength threshold for providing a pathogen positive test result based on the CMYK values. The one or more processors can be configured to identify whether the color wavelength data meets or exceeds the wavelength threshold for providing a pathogen positive test result based on the CMYK values. The one or more processors can be configured to generate a result indicator indicating either a pathogen positive or pathogen negative test result based on the CMYK values.

In another aspect, the sensor can comprise a white light emitter, and a light receiver having a 540 nm filter. In one aspect, the white light emitter can be configured to emit white light towards the pathogen test. In one example, the reflected light intensity from a reaction layer of the pathogen test can be detected using a light receiver having a 540 nm filter. In another example, the reflected light intensity from the reaction layer from the pathogen test can be detected using a light receiver having a filter configured to detect a wavelength in a testing range for the pathogen test.

In yet another aspect, the sensor can comprise one or more of: a photoconductive sensor, a photovoltaic sensor, a photodiode sensor, a phototransistor sensor, or combinations thereof. In another example, the sensor can comprise one or more of a photoresistor, a photodiode array, a charge-coupled device (CCD) camera, a complementary metal-oxide semiconductor (CMOS) camera, the like, or combinations thereof.

In yet another aspect, the system can further comprise a graphical user interface for interacting with a user. The graphical user interface can include a display screen configured to display: (i) an inconclusive test result, (b) the pathogen negative test result, or (c) the pathogen positive test result. In another example, the display screen can be configured to display other aspects of the tests results including one or more of the color wavelength data, the wavelength threshold, the material group identifier, the confidence level, the number of nucleic acids, the pathogens that are negative or positive, the like, or combinations thereof.

In one example, the pathogen can comprise a viral pathogen, a bacterial pathogen, a fungal pathogen, or a protozoa pathogen. In one aspect, the pathogen can comprise a viral pathogen. In one example, the viral pathogen can comprise a dsDNA virus, an ssDNA virus, a dsRNA virus, a positive-strand ssRNA virus, a negative-strand ssRNA virus, an ssRNA-RT virus, or a ds-DNA-RT virus. In one example, each primer sequence can match a sequence from a viral target comprising H1N1, H2N2, H3N2, H1N1pdm09, or SARS-CoV-2.

In another aspect, instead of a pathogen, the specific target nucleotide sequences to be detected can be target nucleotides corresponding to human biomarkers. Any disease that has a target nucleotide corresponding to a human biomarker for a disease can be detected. Various types of diseases can be detected including one or more of: breast cancer, pancreatic cancer, colorectal cancer, ovarian cancer, gastrointestinal cancer, cervix cancer, lung cancer, bladder cancer, many types of carcinomas, salivary gland cancer, kidney cancer, liver cancer, lymphoma, leukemia, melanoma, prostate cancer, thyroid cancer, stomach cancer, the like, or combinations thereof. For example, biomarkers for various types of diseases can be detected by detecting target nucleotides corresponding to one or more of: alpha fetoprotein, CA15-3 and CA27-29, CA19-9, C!-125, calcitonin, calretinin, carcinoembryonic antigen, CD34, CD99MIC 2, CD117, chromogranin, chromosomes 3, 7, 17, and 9p21, cytokeratin, cesmin, epithelial membrane antigen, factor VIII, CD31 FL1, glial fibrillary acidic protein, gross cystic disease fluid protein, hPG80, HMB-45, human chorionic gonadotropin, immunoglobulin, inhibin, keratin, lymphocyte marker, MART-1, Myo D1, muscle-specific actin, neurofilament, neuron-specific enolase, placental alkaline phosphatase, prostate-specific antigen, PTPRC, S100 protein, smooth muscle action, synaptophysin, thymidine kinase, thyroglobulin, thyroid transcription factor-1, tumor M2-PK, vimentin, the like, or combinations thereof.

In another embodiment, a method of identifying a test result of a loop-mediated isothermal amplification (LAMP) reaction on a solid phase reaction medium can comprise detecting, using a sensor component, a spectrum of color wavelengths. In one aspect, the method can comprise receiving, at one or more processors, color wavelength data from the sensor component. In another aspect, the method can comprise determining, at the one or more processors, whether the color wavelength data meets or exceeds the wavelength threshold for providing a positive test result. In another aspect, the method can comprise generating, at the one or more processors, a result indicator indicating either a positive or negative test result. In another aspect, the method can comprise displaying, at a user interface, a test result based on the result indicator.

In another aspect, the method can comprise adjusting, at the one or more processors, the wavelength threshold using RGB values or CMYK values from a material group identifier. In another aspect, the method can comprise receiving, at the one or more processors, RGB values or CMYK values from the spectrum of color wavelengths. In another aspect, the method can comprise generating, at the one or more processors, the result indicator indicating either the positive or negative test result based on the RGB or CMYK values.

In yet another embodiment, at least one machine readable storage medium can have instructions embodied thereon for identifying a test result of a loop-mediated isothermal amplification (LAMP) reaction, wherein the instructions when executed by one or more processors can perform the following: receiving, at the one or more processors, color wavelength data from a sensor component. In another aspect, the instructions when executed can perform: determining, at the one or more processors, whether the color wavelength data exceeds a wavelength threshold for providing a positive test result. In another aspect, the instructions when executed can perform: generating, at the one or more processors, a result indicator indicating either a positive or negative test result. In another aspect, the instructions when executed can perform adjusting the wavelength threshold based on a material group identifier.

In another aspect, the material group identifier can be based on one or more of: an average color wavelength of a material group, a median color wavelength of the material group, a variance of the color wavelength of the material group, a manufacturing date and time of the material group, one or more reagent types of the material group, or one or more solid-phase reaction medium types of the material group. In another aspect, the instructions when executed can perform generating the material group identifier from color wavelength data aggregated from crowd-sourced data. In another aspect, the instructions when executed can perform calculating a number of nucleic acid copies based on the color wavelength data and the material group identifier. In another aspect, the instructions when executed can perform generating a confidence level using the color wavelength data and the material group identifier.

In another aspect, the instructions when executed can perform: generating, at the one or more processors, a result indicator indicating either a positive or negative test result for a plurality of pathogens based on color wavelength data received for discrete sections of the pathogen test.

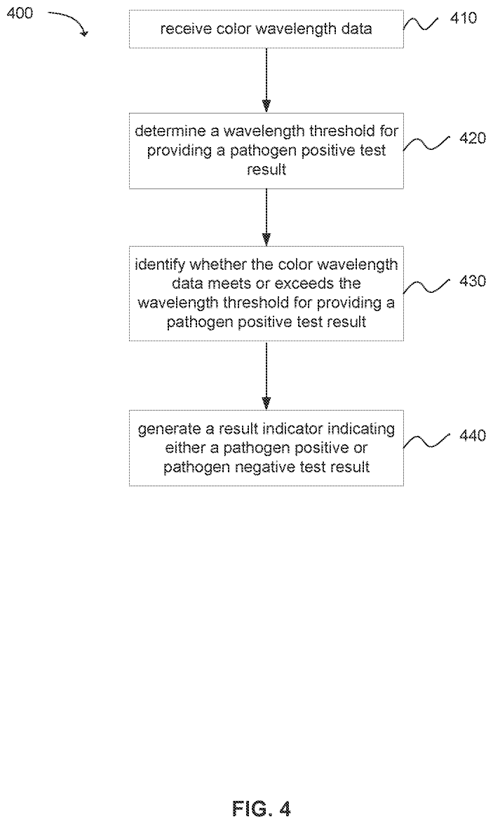

Another example provides functionality 400 of a system for identifying a colorimetric test result from a pathogen test performed on a solid phase substrate, as shown in the flow chart in FIG. 4. The system can comprise a sensor configured to detect a spectrum of color wavelengths. The system can further comprise one or more processors. The one or more processors can be configured to receive color wavelength data, as shown in block 410. The one or more processors can be configured to determine a wavelength threshold for providing a pathogen positive test result, as shown in block 420. The one or more processors can be configured to identify whether the color wavelength data meets or exceeds the wavelength threshold for providing a pathogen positive test result, as shown in block 430. The one or more processors can be configured to generate a result indicator indicating either a pathogen positive or pathogen negative test result, as shown in block 440.

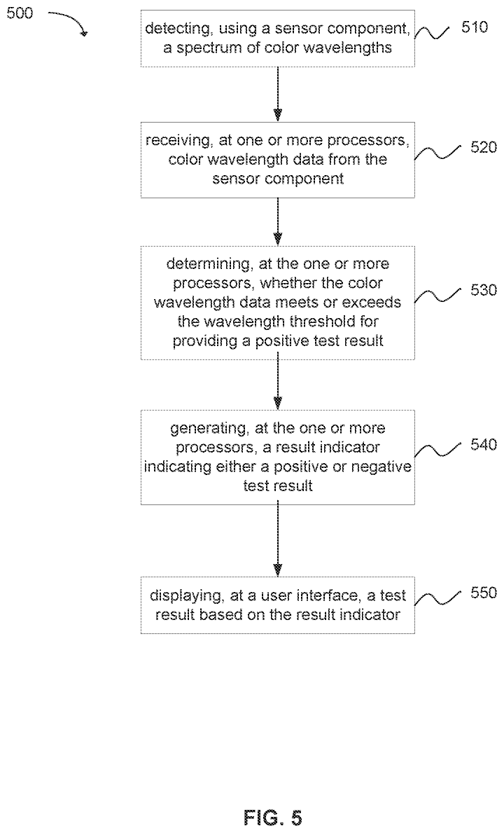

Another example provides a method 500 of identifying a test result of a loop-mediated isothermal amplification (LAMP) reaction on a solid phase reaction medium, as shown in the flow chart in FIG. 5. The method can comprise detecting, using a sensor component, a spectrum of color wavelengths, as shown in block 510. The method can comprise receiving, at one or more processors, color wavelength data from the sensor component, as shown in block 520. The method can comprise determining, at the one or more processors, whether the color wavelength data meets or exceeds the wavelength threshold for providing a positive test result, as shown in block 530. The method can comprise generating, at the one or more processors, a result indicator indicating either a positive or negative test result, as shown in block 540. The method can comprise displaying, at a user interface, a test result based on the result indicator, as shown in block 550.

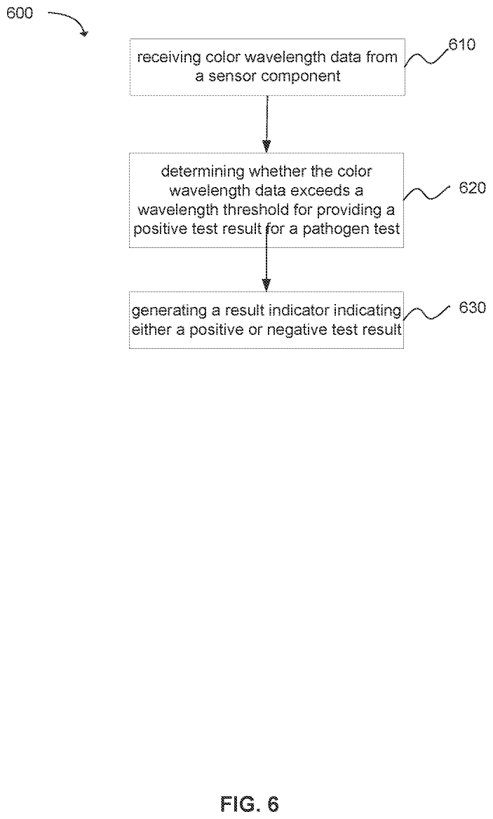

Another example provides at least one machine readable storage medium having instructions 600 embodied thereon for identifying a test result of a loop-mediated isothermal amplification (LAMP) reaction, as shown in FIG. 6. The instructions can be executed on a machine, where the instructions are included on at least one computer readable medium or one non-transitory machine readable storage medium. The instructions when executed can perform: receiving, at the one or more processors, color wavelength data from a sensor component, as shown in block 610. The instructions when executed can perform: determining, at the one or more processors, whether the color wavelength data exceeds a wavelength threshold for providing a positive test result, as shown in block 620. The instructions when executed can perform: generating, at the one or more processors, a result indicator indicating either a positive or negative test result, as shown in block 630.

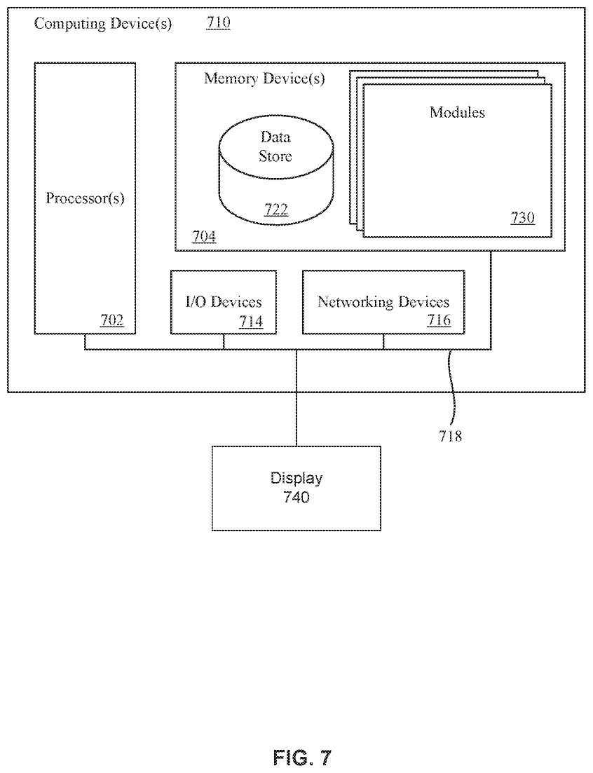

FIG. 7 illustrates a general computing system or device 700 that can be employed in the present technology. The computing system 700 can include a processor 702 in communication with a memory 704. The memory 704 can include any device, combination of devices, circuitry, and the like that is capable of storing, accessing, organizing, and/or retrieving data. Non-limiting examples include SANs (Storage Area Network), cloud storage networks, volatile or non-volatile RAM, phase change memory, optical media, hard-drive type media, and the like, including combinations thereof.

The computing system or device 700 additionally includes a local communication interface 718 for connectivity between the various components of the system. For example, the local communication interface can be a local data bus and/or any related address or control busses as may be desired.

The computing system or device 700 can also include an I/O (input/output) interface 714 for controlling the I/O functions of the system, as well as for I/O connectivity to devices outside of the computing system 700. A network interface 716 can also be included for network connectivity. The network interface 716 can control network communications both within the system and outside of the system. The network interface can include a wired interface, a wireless interface, a Bluetooth interface, optical interface, and the like, including appropriate combinations thereof. Furthermore, the computing system 700 can additionally include a user interface 714, a display device 740, as well as various other components that would be beneficial for such a system.

The processor 702 can be a single or multiple processors, and the memory 704 can be a single or multiple memories. The local communication interface 718 can be used as a pathway to facilitate communication between any of a single processor, multiple processors, a single memory, multiple memories, the various interfaces, and the like, in any useful combination.

### EXAMPLES

In one example there is provided, a system for identifying a colorimetric test result from a pathogen test performed on a solid phase substrate that can comprise a sensor configured to detect a spectrum of color wavelengths; and one or more processors configured to: receive color wavelength data; determine a wavelength threshold for providing a pathogen positive test result; identify whether the color wavelength data meets or exceeds the wavelength threshold for providing a pathogen positive test result; and generate a result indicator indicating either a pathogen positive or pathogen negative test result.

In one example of a system for identifying a colorimetric test result from a pathogen test performed on a solid phase substrate, the one or more processors can be further configured to: adjust the wavelength threshold based on a material group identifier.

In another example of a system for identifying a colorimetric test result from a pathogen test performed on a solid phase substrate, the material group identifier can be based on one or more of: an average color wavelength of a material group, a median color wavelength of the material group, a variance of the color wavelength of the material group, a manufacturing date and time of the material group, one or more reagent types of the material group, or one or more solid-phase reaction medium types of the material group.

In another example of a system for identifying a colorimetric test result from a pathogen test performed on a solid phase substrate, the one or more processors can be further configured to: generate the material group identifier from color wavelength data aggregated from crowd-sourced data.

In another example of a system for identifying a colorimetric test result from a pathogen test performed on a solid phase substrate, the one or more processors can be further configured to: calculate a number of nucleic acid copies based on one or more of the color wavelength data, the material group identifier, or a color change time.

In another example of a system for identifying a colorimetric test result from a pathogen test performed on a solid phase substrate, the one or more processors can be further configured to: generate a confidence level using the color wavelength data and the material group identifier.

In another example of a system for identifying a colorimetric test result from a pathogen test performed on a solid phase substrate, the one or more processors can be further configured to: receive the color wavelength data for discrete sections of the pathogen test; determine the wavelength threshold for providing a pathogen positive test result based on the color wavelength data for discrete sections of the pathogen test; and identify whether the color wavelength data meets or exceeds the threshold for providing the pathogen positive test result.

In another example of a system for identifying a colorimetric test result from a pathogen test performed on a solid phase substrate, the one or more processors can be further configured to generate a confidence level for the pathogen positive test result based on the color wavelength data received from the discrete sections of the pathogen test.

In another example of a system for identifying a colorimetric test result from a pathogen test performed on a solid phase substrate, the one or more processors can be further configured to: receive the color wavelength data for discrete sections of the pathogen test; determine an additional wavelength threshold for providing a different pathogen positive test result based on the color wavelength data for discrete sections of the pathogen test; and generate an additional result indicator indicating either an additional pathogen positive or additional pathogen negative test result when the discrete sections of the pathogen test are targeted to different pathogens.

In another example of a system for identifying a colorimetric test result from a pathogen test performed on a solid phase substrate, the sensor can be configured to detect a spectrum of color wavelengths for discrete sections of the pathogen test.

In another example of a system for identifying a colorimetric test result from a pathogen test performed on a solid phase substrate, the sensor can comprise an RGB sensor configured to generate RGB values or a CMYK sensor configured to generate CMYK values.

In another example of a system for identifying a colorimetric test result from a pathogen test performed on a solid phase substrate, the sensor can comprise: a white light emitter; and a light receiver having a 540 nm filter.

In another example of a system for identifying a colorimetric test result from a pathogen test performed on a solid phase substrate, the one or more processors can be further configured to: adjust the wavelength threshold using color wavelength data having a wavelength from about 500 nm to about 565 nm.

In another example of a system for identifying a colorimetric test result from a pathogen test performed on a solid phase substrate, the sensor can be one or more of: a photoconductive sensor, a photovoltaic sensor, a photodiode sensor, a phototransistor sensor, or combinations thereof.

In another example of a system for identifying a colorimetric test result from a pathogen test performed on a solid phase substrate, the system can further comprise a graphical user interface configured to display the pathogen negative or pathogen positive test result.

In another example there is provided, a method of identifying a test result of a loop-mediated isothermal amplification (LAMP) reaction on a solid phase reaction medium that can comprise: detecting, using a sensor component, a spectrum of color wavelengths; receiving, at one or more processors, color wavelength data from the sensor component; determining, at the one or more processors, whether the color wavelength data meets or exceeds the wavelength threshold for providing a positive test result; generating, at the one or more processors, a result indicator indicating either a positive or negative test result; and displaying, at a user interface, a test result based on the result indicator.

In another example of a method of identifying a test result of a loop-mediated isothermal amplification (LAMP) reaction on a solid phase reaction medium, the method can further comprise adjusting, at the one or more processors, the wavelength threshold using RGB values or CMYK values from a material group identifier.

In another example of a method of identifying a test result of a loop-mediated isothermal amplification (LAMP) reaction on a solid phase reaction medium, the method can further comprise receiving, at the one or more processors, RGB values or CMYK values from the spectrum of color wavelengths.

In another example of a method of identifying a test result of a loop-mediated isothermal amplification (LAMP) reaction on a solid phase reaction medium, the method can further comprise generating, at the one or more processors, the result indicator indicating either the positive or negative test result based on the RGB or CMYK values.

In another example of a method of identifying a test result of a loop-mediated isothermal amplification (LAMP) reaction on a solid phase reaction medium, at least one machine readable storage medium having instructions embodied thereon for identifying a test result of a loop-mediated isothermal amplification (LAMP) reaction, the instructions when executed by one or more processors can perform the following: receiving color wavelength data from a sensor component; determining whether the color wavelength data exceeds a wavelength threshold for providing a positive test result for a pathogen test; and generating a result indicator indicating either a positive or negative test result.

In another example of a method of identifying a test result of a loop-mediated isothermal amplification (LAMP) reaction on a solid phase reaction medium, the method can further comprise instructions that when executed perform: adjusting the wavelength threshold based on a material group identifier.

In another example of a method of identifying a test result of a loop-mediated isothermal amplification (LAMP) reaction on a solid phase reaction medium, the material group identifier is based on one or more of: an average color wavelength of a material group, a median color wavelength of the material group, a variance of the color wavelength of the material group, a manufacturing date and time of the material group, one or more reagent types of the material group, or one or more solid-phase reaction medium types of the material group.

In another example of a method of identifying a test result of a loop-mediated isothermal amplification (LAMP) reaction on a solid phase reaction medium, further comprising instructions that when executed perform: generating the material group identifier from color wavelength data aggregated from crowd-sourced data.

In another example of a method of identifying a test result of a loop-mediated isothermal amplification (LAMP) reaction on a solid phase reaction medium, the method can further comprise instructions that when executed perform: calculating a number of nucleic acid copies based on the color wavelength data and the material group identifier.

In another example of a method of identifying a test result of a loop-mediated isothermal amplification (LAMP) reaction on a solid phase reaction medium, the method can further comprise instructions that when executed perform: generating a confidence level using the color wavelength data and the material group identifier.

In another example of a method of identifying a test result of a loop-mediated isothermal amplification (LAMP) reaction on a solid phase reaction medium, the method can further comprise instructions that when executed perform: generating a result indicator indicating either a positive or negative test result for a plurality of pathogens based on color wavelength data received for discrete sections of the pathogen test.

Various techniques, or certain aspects or portions thereof, can take the form of program code (i.e., instructions) embodied in tangible media, such as floppy diskettes, compact disc-read-only memory (CD-ROMs), hard drives, non-transitory computer readable storage medium, or any other machine-readable storage medium wherein, when the program code is loaded into and executed by a machine, such as a computer, the machine becomes an apparatus for practicing the various techniques. Circuitry can include hardware, firmware, program code, executable code, computer instructions, and/or software. A non-transitory computer readable storage medium can be a computer readable storage medium that does not include signal. In the case of program code execution on programmable computers, the computing device can include a processor, a storage medium readable by the processor (including volatile and non-volatile memory and/or storage elements), at least one input device, and at least one output device. The volatile and non-volatile memory and/or storage elements can be a random-access memory (RAM), erasable programmable read only memory (EPROM), flash drive, optical drive, magnetic hard drive, solid state drive, or other medium for storing electronic data. The low energy fixed location node, wireless device, and location server can also include a transceiver module (i.e., transceiver), a counter module (i.e., counter), a processing module (i.e., processor), and/or a clock module (i.e., clock) or timer module (i.e., timer). One or more programs that can implement or utilize the various techniques described herein can use an application programming interface (API), reusable controls, and the like. Such programs can be implemented in a high level procedural or object oriented programming language to communicate with a computer system. However, the program(s) can be implemented in assembly or machine language, if desired. In any case, the language can be a compiled or interpreted language, and combined with hardware implementations.

As used herein, the term processor can include general purpose processors, specialized processors such as VLSI, FPGAs, or other types of specialized processors, as well as base band processors used in transceivers to send, receive, and process wireless communications.

It should be understood that many of the functional units described in this specification have been labeled as modules, in order to more particularly emphasize their implementation independence. For example, a module can be implemented as a hardware circuit comprising custom very-large-scale integration (VLSI) circuits or gate arrays, off-the-shelf semiconductors such as logic chips, transistors, or other discrete components. A module can also be implemented in programmable hardware devices such as field programmable gate arrays, programmable array logic, programmable logic devices or the like.

In one example, multiple hardware circuits or multiple processors can be used to implement the functional units described in this specification. For example, a first hardware circuit or a first processor can be used to perform processing operations and a second hardware circuit or a second processor (e.g., a transceiver or a baseband processor) can be used to communicate with other entities. The first hardware circuit and the second hardware circuit can be incorporated into a single hardware circuit, or alternatively, the first hardware circuit and the second hardware circuit can be separate hardware circuits.

Modules can also be implemented in software for execution by various types of processors. An identified module of executable code can, for instance, comprise one or more physical or logical blocks of computer instructions, which can, for instance, be organized as an object, procedure, or function. Nevertheless, the executables of an identified module need not be physically located together, but can comprise disparate instructions stored in different locations which, when joined logically together, comprise the module and achieve the stated purpose for the module.

Indeed, a module of executable code can be a single instruction, or many instructions, and can even be distributed over several different code segments, among different programs, and across several memory devices. Similarly, operational data can be identified and illustrated herein within modules, and can be embodied in any suitable form and organized within any suitable type of data structure. The operational data can be collected as a single data set, or can be distributed over different locations including over different storage devices, and can exist, at least partially, merely as electronic signals on a system or network. The modules can be passive or active, including agents operable to perform desired functions.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

What is claimed is:

1. A system for identifying a colorimetric test result from a target nucleotide test performed on a solid phase substrate comprising:
a sensor configured to detect a spectrum of color wavelengths; and
one or more processors configured to:
receive color wavelength data;
determine a wavelength threshold for providing a target nucleotide positive test result;
identify whether the color wavelength data meets or exceeds the wavelength threshold for providing a target nucleotide positive test result; and
generate a result indicator indicating either a target nucleotide positive test result or a target nucleotide negative test result;
wherein the one or more processors are further configured to:
receive the color wavelength data for discrete sections of the target nucleotide;
determine an additional wavelength threshold for providing a different target nucleotide positive test result based on the color wavelength data for discrete sections of the target nucleotide test; and
generate an additional result indicator indicating either an additional target nucleotide positive or additional target nucleotide negative test result when the discrete sections of the target nucleotide test are targeted to different target nucleotides.

2. The system of claim 1, wherein the one or more processors are further configured to:
adjust the wavelength threshold based on a material group identifier.

3. The system of claim 2, wherein the material group identifier is based on one or more of:
an average color wavelength of a material group,
a median color wavelength of the material group,
a variance of the color wavelength of the material group,
a manufacturing date and time of the material group,
one or more reagent types of the material group, or
one or more solid-phase reaction medium types of the material group.

4. The system of claim 2, wherein the one or more processors are further configured to:
generate the material group identifier from color wavelength data aggregated from crowd-sourced data.

5. The system of claim 2, wherein the one or more processors are further configured to:
calculate a number of nucleic acid copies based on one or more of the color wavelength data, the material group identifier, or a color change time.

6. The system of claim 2, wherein the one or more processors are further configured to:
generate a confidence level using the color wavelength data and the material group identifier.

7. The system of claim 1, wherein the one or more processors are further configured to:
generate a confidence level for the target nucleotide positive test result based on the color wavelength data received from the discrete sections of the target nucleotide test.

8. The system of claim 1, wherein the sensor comprises an RGB sensor configured to generate RGB values or a CMYK sensor configured to generate CMYK values.

9. The system of claim 1, wherein the sensor comprises:
a white light emitter; and
a light receiver having a 540 nm filter.

10. The system of claim 1, wherein the one or more processors are further configured to:
adjust the wavelength threshold using color wavelength data having a wavelength from about 500 nm to about 565 nm.

11. The system of claim 1, wherein the sensor is one or more of: a photoconductive sensor, a photovoltaic sensor, a photodiode sensor, a phototransistor sensor, or combinations thereof.

12. The system of claim 1, further comprising a graphical user interface configured to display the target nucleotide negative test result or the target nucleotide positive test result.

13. The system of claim 1, wherein:
the target nucleotide corresponds to a microorganism, antimicrobial resistance gene, trait, or a biomarker associated with physiology or pathology, and
the microorganism comprises a bacterium, a virus, a fungus, or a protozoan.

14. A method of identifying a test result of a loop-mediated isothermal amplification (LAMP) reaction on a solid phase reaction medium, comprising:
detecting, using a sensor component, a spectrum of color wavelengths;
receiving, at one or more processors, color wavelength data from the sensor component;
determining, at the one or more processors, whether the color wavelength data meets or exceeds a wavelength threshold for providing a positive test result;
generating, at the one or more processors, a result indicator indicating either a positive or negative test result;
displaying, at a user interface, a test result based on the result indicator; receiving, at the one or more processors, the color wavelength data for discrete sections of the solid phase reaction medium;
determining, at the one or more processors, an additional wavelength threshold for providing a different target nucleotide positive test result based on the color wavelength data for discrete sections of the solid phase reaction medium; and
generating, at the one or more processors, an additional result indicator indicating either an additional target nucleotide positive test result or an additional target nucleotide negative test result when the discrete sections of the solid phase reaction medium are targeted to different target nucleotides.

15. The method of claim 14, further comprising:
adjusting, at the one or more processors, the wavelength threshold using RGB values or CMYK values from a material group identifier.

16. The method of claim 14, further comprising:
receiving, at the one or more processors, RGB values or CMYK values from the spectrum of color wavelengths; and
generating, at the one or more processors, the result indicator indicating either the additional target nucleotide positive test result or the additional target nucleotide negative test result based on the RGB or CMYK values.

17. The method of claim 14, wherein:
the target nucleotide corresponds to a microorganism, antimicrobial resistance gene, trait, or a biomarker associated with physiology or pathology, and
the microorganism comprises a bacterium, a virus, a fungus, or a protozoan.
